(19) United States
(12) Patent Application Publication
(10) Pub. No.: US 2008/0281364 A1
(43) Pub. Date: Nov. 13, 2008

(54) SYSTEMS, DEVICES AND METHODS FOR STABILIZING BONE

(75) Inventors: Paul E. Chirico, Campbell, CA (US); Benny M. Chan, Fremont, CA (US); Gary B. Hulme, San Jose, CA (US); Jeffrey J. Christian, Morgan Hill, CA (US)

Correspondence Address: SHAY GLENN LLP, 2755 CAMPUS DRIVE, SUITE 210, SAN MATEO, CA, 94403, US

(73) Assignee: SPINEWORKS MEDICAL, INC., San Jose, CA (US)

(21) Appl. No.: 12/024,938

(22) Filed: Feb. 1, 2008

Related U.S. Application Data
(60) Provisional application No. 60/916,731, filed on May 8, 2007.

(51) Int. Cl. A61F 5/00; A61B 17/70; A61M 29/00

(52) U.S. Cl. 606/86 A; 606/246; 606/191

(57) ABSTRACT

Described herein are devices, systems, and methods for treating bone, particularly for restoring bone dimension in compression fractures. Self-expanding stabilization devices for repairing bone may include two or more continuous curvature of bending struts that extend from a central shaft in the deployed configuration. The stabilization device may be attached to an inserter. An inserter may be used to hold the stabilization device in a collapsed delivery configuration so that it can be inserted into bone. In use, the stabilization device may be part of a system or kit for installing the device into a bone region and allowing it to expand to correct a bone fracture.

### CROSS REFERENCE TO RELATED APPLICATIONS

This application claims priority to U.S. Provisional Patent Application Ser. No. 60/916,731, titled “SYSTEMS, DEVICES AND METHODS FOR STABILIZING BONE,” filed on May 8, 2007.

This application is related to U.S. patent application Ser. No. 11/468,759, filed Aug. 30, 2006, which claims the benefit of U.S. Provisional Application No. 60/713,259, filed Aug. 31, 2005. All of these applications are incorporated herein by reference in their entirety.

### INCORPORATION BY REFERENCE

All publications and patent applications mentioned in this specification are herein incorporated by reference to the same extent as if each individual publication or patent application was specifically and individually indicated to be incorporated by reference, in their entirety.

### FIELD OF THE INVENTION

Described herein are systems, devices, and methods for treating and supporting bone within a skeletal structure. The invention also relates to systems, devices, and methods for treating and supporting cancellous bone within vertebral bodies, particularly vertebral bodies which have suffered a vertebral compression fracture (VCF).

### BACKGROUND OF THE INVENTION

Deterioration of bone tissue, and particularly micro-architecture deterioration, can result from a variety of factors including disease, aging, stress and use. For example, osteoporosis is a disease characterized by low bone mass and micro-architecture deterioration of bone tissue. Osteoporosis leads to bone fragility and an increase fracture risk. The World Health Organization defines osteoporosis as a bone density more than 2.5 standard deviations below the young adult mean value. Values between 1 and 2.5 standard deviation below the young adult mean are referred to as osteopenia.

While osteoporosis affects the entire skeleton, it commonly causes fractures in the spine and hip. Spinal or vertebral fractures have serious consequences, with patients suffering from loss of height, deformity, and persistent pain that can significantly impair mobility and quality of life. An estimated 1.5 million elderly people in the United States suffer an osteoporotic fracture each year. Of these fractures, an estimated 750,000 are vertebral compression fractures (VCFs) and 250,000 are hip fractures. VCFs in women age 50 and older is estimated to be greater than 25%, with the rate increasing with age. Fracture pain usually lasts 4 to 6 weeks, with intense pain at the fracture site.

In an osteoporotic bone, pores or voids in the sponge-like cancellous bone increase in dimension, making the bone very fragile. Although bone breakdown occurs continually as the result of osteoclast activity in young, healthy bone tissue, this breakdown is balanced by new bone formation by osteoblasts. In contrast, in an elderly patient, bone resorption can surpass bone formation, resulting in deterioration of bone density. Osteoporosis occurs largely without symptoms until a fracture occurs.

While there have been pharmaceutical advances aimed toward slowing or arresting bone loss, new and improved solutions to treating VCFs are still needed as the number of people suffering from VCFs is predicted to grow steadily as life expectancy increases.

As illustrated in FIG. 1A, the spine includes a plurality of vertebral bodies with intervening intervertebral discs. Both the width and depth of the vertebral bodies increase as the spine descends in the rostral-to-caudal direction. The height of the vertebral bodies also increase in the rostral-to-caudal direction, with the exception of a slight reversal at C6 and lower lumbar levels.

Vertebra, as well as other skeletal bones, are made up of a thick cortical shell and an inner meshwork of porous cancellous bone. Cancellous bone is comprised of collagen, calcium salts and other minerals. Cancellous bone also has blood vessels and bone marrow in the spaces.

Vertebroplasty and kyphoplasty are recently developed techniques for treating vertebral compression fractures. Percutaneous vertebroplasty was first reported in 1987 for the treatment of hemangiomas. In the 1990's, percutaneous vertebroplasty was extended to indications including osteoporotic vertebral compression fractures, traumatic compression fractures, as well as vertebral metastasis. In one percutaneous vertebroplasty technique, bone cement such as PMMA (polymethylmethacrylate) is percutaneously injected into a fractured vertebral body through a trocar and cannula system. The targeted vertebrae are identified under fluoroscopy, and a needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement typically contains radiopaque materials so that when injected under live fluoroscopy, cement localization and leakage can be observed. The visualization of PMMA injection and extravasion are critical to the technique and the physician terminates PMMA injection when leakage is evident. The cement is injected using small syringe-like injectors to allow the physician to manually control the injection pressures.

Kyphoplasty is a modification of percutaneous vertebroplasty in which a void is created mechanically by compression. Balloon kyphoplasty involves a preliminary step that comprises the percutaneous placement of an inflatable balloon tamp in the vertebral body. Inflation of the balloon creates a cavity in the bone prior to cement injection. It is unclear if percutaneous kyphoplasty using a high pressure balloon-tamp inflation can at least partially restore vertebral body height. In balloon kyphoplasty, it has been proposed that PMMA can be injected at lower pressures into the collapsed vertebra since a cavity exists within the vertebral body to receive the cement—which is not the case in conventional vertebroplasty.

The principal indications for any form of vertebroplasty are osteoporotic vertebral collapse with debilitating pain. Often, radiography and computed tomography are performed in the days preceding treatment to determine the extent of vertebral collapse, the presence of epidural or foraminal stenosis caused by bone fragment retropulsion, the presence of cortical destruction or fracture and the visibility, and degree of involvement of the pedicles. Leakage of PMMA during vertebroplasty and/or kyphoplasty can result in very serious complications including compression of adjacent structures that necessitate emergency decompressive surgery.

The human spinal column 10, as shown in FIG. 1A, is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

An example of one vertebra is illustrated in FIG. 1B, which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles, extend dorsally from each side of the vertebral body 14 to form a vertebral arch 18 which defines the vertebral foramen.

At the posterior end of each pedicle 25, the vertebral arch 18 flares out into broad plates of bone known as the laminae 20. The laminae 20 fuse with each other to form a spinous p 22. The spinous p 22 provides for muscle and ligamentous attachment. A smooth transition from the pedicles to the laminae 20 is interrupted by the formation of a series of pes. Two transverse pes thrust out laterally, one on each side, from the junction of the pedicle with the lamina 20. The transverse pes serve as levers for the attachment of muscles to the vertebrae 12. Four articular pes, two superior and two inferior, also rise from the junctions of the pedicles and the laminae 20. The superior articular pes are sharp oval plates of bone rising upward on each side of the vertebrae, while the inferior pes 28, 28′ are oval plates of bone that jut downward on each side.

The superior and inferior articular pes each have a natural bony structure known as a facet. The superior articular facet faces medially upward, while the inferior articular facet faces laterally downward. When adjacent vertebrae 12 are aligned, the facets, capped with a smooth articular cartilage and encapsulated by ligaments, interlock to form a facet joint 32. The facet joints are apophyseal joints that have a loose capsule and a synovial lining.

An intervertebral disc 34 between each adjacent vertebra 12 (with stacked vertebral bodies shown as 14, 15 in FIG. 1C) permits gliding movement between the vertebrae 12. The structure and alignment of the vertebrae 12 thus permit a range of movement of the vertebrae 12 relative to each other. FIG. 1D illustrates a posterolateral oblique view of a vertebra 12. The vertebral body 14 is shown in a cut-away that illustrates the cortical bone 40 which forms the exterior of the bone (in this case the vertebral body) and the spongy cancellous bone 42 located within the interior of the cortical bone.

Despite the small differences in mineralization, the chemical composition and true density of cancellous bone are similar to those of cortical bone. As a result, the classification of bone tissue as either cortical or cancellous is based on bone porosity, which is the proportion of the volume of bone occupied by non-mineralized tissue. Cortical bone has a porosity of approximately 5-30% whereas cancellous bone porosity may range from approximately 30 to more than 90%. Although typically cortical bone has a higher density than cancellous bone, that is not necessarily true in all cases. As a result, for example, the distinction between very porous cortical bone and very dense cancellous bone can be somewhat arbitrary.

The mechanical strength of cancellous bone is well known to depend on its apparent density and the mechanical properties have been described as those similar to man-made foams. Cancellous bone is ordinarily considered as a two-phase composite of bone marrow and hard tissue. The hard tissue is often described as being made of trabecular “plates and rods.” Cancellous microstructure can be considered as a foam or cellular solid since the solid fraction of cancellous bone is often less than 20% of its total volume and the remainder of the tissue (marrow) is ordinarily not significantly load carrying. The experimental mechanical properties of trabecular tissue samples are similar to those of many man-made foams. If a sample of tissue is crushed under a prescribed displacement protocol, the load-displacement curve will initially be linear, followed by an abrupt nonlinear “collapse” where the load carrying capacity of the tissue is reduced by damage. Next follows a period of consolidation of the tissue where the load stays essentially constant, terminated by a rapid increase in the load as the tissue is compressed to the point where the void space is eliminated. Each of the mechanical properties of cancellous bone varies from site-to-site in the body. The apparent properties of cancellous bone as a structure depend upon the conformation of the holes and the mechanical properties of the underlying hard tissue composing the trabeculae. The experimental observation is that the mechanical properties of bone specimens are power functions of the solid volume fraction. The microstructural measures used to characterize cancellous bone are very highly correlated to the solid volume fraction. This suggests that the microstructure of the tissue is a single parameter function of solid volume fraction. If this is true, the hard tissue mechanical properties will play a large role in determining the apparent properties of the tissue. At this time, little is known about the dependence of trabecular hard tissue mechanical properties on biochemical composition or ultrastructural organization.

Cancellous bone in the joints and spine is continuously subject to significant loading. One consequence of this is that the tissue can experience, and occasionally accumulate, microscopic fractures and cracks. These small damages are similar to those seen in man-made materials and are, in many cases, the result of shear failure of the material. It is known that microcracks accumulate with age in the femoral head and neck, leading to a hypothesis that these damages are related to the increase in hip fracture with age. However, no such association of increased crack density with age was found in human vertebral cancellous bone despite the high incidence of spinal fractures, particularly in women.

Adult cortical and cancellous bone can be considered as a single material whose apparent density varies over a wide range. The compressive strength of bone tissue is proportional to the square of the apparent density. Cortical bone morphology and composition can be characterized by an examination of microstructure, porosity, mineralization, and bone matrix. These parameters seldom vary independently but are usually observed to vary simultaneously. Mechanical properties vary through the cortical thickness due to variations in microstructure, porosity, and chemical composition.

Mechanical properties are dependent on microstructure. The strongest bone type is circumferential lamellar bone, followed in descending order of strength by primary laminar, secondary Haversian, and woven-fibered bone. All normal adult cortical bone is lamellar bone. Most of the cortical thickness is composed of secondary Haversian bone. Circumferential lamellar bone is usually present at the endosteal and periosteal surfaces. In the adult, woven-fibered bone is formed only during rapid bone accretion, which accompanies conditions such as fracture callus formation, hyperparathyroidism, and Paget's disease.

Aging is associated with changes in bone microstructure which are caused primarily by internal remodeling throughout life. In the elderly, the bone tissue near the periosteal surface is stronger and stiffer than that near the endosteal surface due primarily to the porosity distribution through the cortical thickness caused by bone resorption. Bone collagen intermolecular cross-linking and mineralization increase markedly from birth to 17 years of age and continue to increase, gradually, throughout life. Adult cortical bone is stronger and stiffer and exhibits less deformation to failure than bone from children. Cortical bone strength and stiffness are greatest between 20 and 39 years of age. Further aging is associated with a decrease in strength, stiffness, deformation to failure, and energy absorption capacity.

From this understanding of bone, it can be appreciated that when a vertebral body becomes damaged, as illustrated in FIG. 1E, such as when a fracture 80 occurs, a portion of the vertebral body typically collapses. This collapse can occur as a result of micro-architecture deterioration of the bone tissue.

The terms caudal and cephalad may be used in conjunction with the devices and operation of the devices and tools herein to assist in understanding the operation and/or position of the device and/or tools.

In order to understand the configurability, adaptability, and operational aspects of the invention disclosed herein, it is helpful to understand the anatomical references of the body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 1F). Additionally, devices and the operation of devices and tools are better understood with respect to the caudad 60 direction and/or the cephalad direction 62. Devices and tools can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the devices, systems and tools of the present invention may be configurable and variable with respect to a single anatomical plane or with respect to two or more anatomical planes. For example, a component may be described as lying within and having adaptability or operability in relation to a single plane. For example, a device may be positioned in a desired location relative to an axial plane and may be moveable between a number of adaptable positions or within a range of positions. Similarly, the various components can incorporate differing sizes and/or shapes in order to accommodate differing patient sizes and/or anticipated loads.

### SUMMARY OF THE INVENTION

Described herein are devices, systems and method for stabilizing a bone, such as a vertebra. In general, the devices for stabilizing bone may include an elongate shaft having two or more struts that are configured to extend from the shaft. The struts are configured to translate between a delivery (e.g., collapsed) configuration into a deployed (e.g., extended) configuration. The struts typically have a continuous curvature of bending. For example, the struts may be hingeless struts or notchless struts. Bone (e.g., non-cancellous bone) may be supported by the struts after the device has been inserted and allowed to expand into cancellous bone. A cement (e.g., a bone cement such as PMMA) may also be used with the implants described herein in order to provide long-term or enhanced strength and stability.

Struts having a continuous curvature of bending (e.g., hingeless or notchless struts) are shown and described in greater detail in some of the figures described below, and are usually configured so that they translate between a delivery and a deployed configuration by bending over the length of the strut rather than by bending at a discrete portion (e.g., at a notch, hinge, channel, or the like). Thus, bending occurs continuously over the length of the strut (e.g., continuously over the entire length of the strut, continuously over the majority of the length of the strut (e.g., between 100-90%, 100-80%, 100-70%, etc.), continuously over approximately half the length of the strut (e.g., between about 60-40%, approximately 50%, etc.). Struts having a continuous curvature of bending are referred to as “continuous curvature of bending struts”.

Many of the stabilization devices described herein have a compressed delivery configuration (or profile) and an expanded deployed configuration (or profile) in which the struts are at least partially extended from the long axis of the device shaft. These devices may be self-expanding form the delivery configuration into the deployed configuration. For example, the devices may be formed so that they are ‘relaxed’ in the deployed configuration, and are held (e.g., in compression) in the delivery configuration; upon release, the device expands into the relaxed deployed configuration. This may be achieved by the use of materials having a sufficient spring constant (e.g., resulting in elastic deformation), or shape memory materials.

In some variations, a stabilization device inserter (or “inserter”) may be used to insert the devices into the bone. In addition, an inserter may be used to hold the device in the delivery configuration, and triggered to allow the device to expand into the deployed configuration. The inserter may also be used to remove the device from the bone. In general, a stabilization device includes attachment sites at either end (distal and proximal) of the stabilization device, and these attachment sits can releasably attach to sites on the inserter. Thus, the inserter is releasably secured to the stabilization device, and can apply force to keep the stabilization device in the compressed delivery configuration by maintaining the separation between the proximal and distal ends of the stabilization device.

For example, in some variations, a stabilization device configured to self-expand from a compressed delivery configuration to an expanded deployed configuration includes an elongate shaft having two or more continuous curvature of bending struts (wherein the struts extend from the shaft more in the deployed configuration than in the delivery configuration), a proximal region having a first releasable attachment configured to attach to an inserter, and a distal region having a second releasable attachment configured to attach to the inserter.

A stabilization device may have two or more slits in the elongate shaft, forming the struts. The stabilization devices described herein may be made of any appropriate material, particularly biocompatible materials. For example, the struts of the device may be formed of a shape memory alloy such as Nitinol.

In some variations, the releasable attachment regions comprise a notch or cut out, into which a peg, slider, or other element from the inserter may mate. For example, the releasable attachment region on the stabilization device may be an L-shaped notch (or J-shaped, S-shaped, etc.) which can mate with a pin on the inserter. Since the stabilization devices typically include two or more releasable attachment regions for mating with the inserter, different releasable attachment regions may be used. For example, a releasable attachment region may be a threaded region that mates with a complementary threaded region on the inserter (e.g., by screwing).

The stabilization deices may be any appropriate dimension for implantation into the body (e.g., bone). For example, the maximum distance between the struts (measured at a point along the length of the shaft) in the expanded deployed configuration can between about 0.5 mm and about 30 mm, about 8 mm and about 20 mm, about 10 mm, about 18 mm, or the like. In some variations, the struts are configured so that the device may be used in a vascular context. For example, the maximum distance between the struts (measured at a point along the length of the shaft) in the expanded deployed configuration may be between about 0.5 mm and about 5 mm.

Also described herein are self-expanding stabilization devices for stabilizing a body cavity. These devices may include an elongate shaft having a plurality of continuous curvature of bending struts extendable there from (the shaft may be adapted to be positioned within cancellous bone) and having an expanded deployed profile and a collapsed delivery profile. The shaft may be adapted to cut through cancellous bone during expansion from the collapsed delivery profile to the expanded deployed profile, and the shaft is also adapted to abut a surface of cortical bone adjacent the cancellous bone without passing there through.

Also described herein are inserters for inserting a stabilization device. An inserter may include a first elongate member having a first stabilization device attachment region that is adapted to releasably attach to the proximal region of the stabilization device, and a second elongate member having a second stabilization device attachment region that is adapted to releasably attach to the distal region of the stabilization device. The second elongate member is axially movable relative to the first elongate member. The first elongate member and the second elongate member may be configured so that they may be independently rotated axially with respect to each other.

As mentioned briefly above, the first and/or the second stabilization device attachment region may include a pin configured to mate with a channel in the proximal region of the stabilization device.

In some variations, the inserter further comprises a handle. Alternatively, or in addition, the inserter may include a knob on the first elongate member. The knob may be used to hold the inserter, or to move (e.g., rotate) the first elongate member, either for retracting/deploying the device, or for releasing the device from the inserter. The handle may include a lock (e.g., a releasable lock) that may be used to secure the position of the handle, and thereby keep the stabilization device compressed (in the delivery configuration), or in the expanded configuration. The handle may also include a release for releasing the stabilization device from the inserter.

In some variations, the second elongate member of the inserter is coaxial to the first elongate member, and may move independently of the first elongate member (e.g., axially or in rotation).

Also described herein are inserters for inserting a stabilization device that include a first elongate member having a stabilization device attachment region at the distal end (wherein the stabilization device attachment region is adapted to releasably attach to the proximal region of the stabilization device), a second elongate member having a stabilization device attachment region at its distal end that is adapted to releasably attach to the distal region of the stabilization device (wherein the second elongate member is axially movable relative to the first elongate member), and the first elongate member and the second elongate member are independently axially rotatable with respect to each other. The inserter may also include a first handle attachment region at the proximal end of the first elongate member and a second handle attachment region at the proximal end of the second elongate member. Thus, the handle may be attached to the inserter by mating with the first and second attachment regions. For example, the handle may be re-usable with different inserters (and different stabilization devices).

Also described herein are systems or kits for stabilizing a vertebral body. These systems or kits may include any of the components described herein, including a stabilization device having an elongate shaft and a plurality of struts extending there from (e.g., a stabilization device configured to expand from a compressed delivery configuration to an expanded deployed configuration), and an inserter having a first stabilization device attachment region that is adapted to releasably secure to the proximal region of the stabilization device and a second stabilization device attachment region adapted to secure to the distal region of the stabilization device.

A system for stabilizing a vertebral body may also include an introducer, handle, trocar, drill (e.g., a twist drill), bone cement, cement cannula, or the like. In general, a system for stabilizing a vertebral body can include any of the stabilization devices and any of the inserters and additional devices or materials described herein.

Also described herein are methods of treating a bone. The methods may include the steps of delivering a self-expanding device within a cancellous bone (wherein the device has an elongate shaft and a plurality of continuous curvature of bending struts extending there from), and allowing the device to expand within the cancellous bone so that a cutting surface of the device cuts through the cancellous bone. The method may also include the steps of visualizing the device within the bone, drilling a hole into the cancellous bone through which the self-expanding device may be inserted, applying force to further expand the device within the cancellous bone, and/or applying bone cement within the cancellous bone.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which.

FIG. 1 is a lateral view of a normal human spinal column. FIG. 1B is a superior view of a normal human lumbar vertebra. FIG. 1C is a lateral view of a functional spinal unit having two vertebral bodies and an intervertebral disc. FIG. 1D is a posterolateral oblique view of a vertebra. FIG. 1E illustrates a portion of a spine wherein a vertebral body is fractured; FIG. 1F illustrates a human body with the planes of the body identified.

FIGS. 2A-2E are variations of stabilization devices.

FIGS. 3A and 3B are enlarged side and side perspective views (respectively) of the stabilization device shown in FIG. 2A.

FIGS. 4A and 4B are enlarged side and side perspective views (respectively) of the stabilization device shown in FIG. 2C.

FIGS. 5A and 5B are enlarged side and side perspective views (respectively) of the stabilization device shown in FIG. 2E.

FIG. 6A is one variation of a stabilization device having a plurality of continuous curvature of bending struts removably attached to an inserter.

FIG. 6B is another variation of a stabilization device removably attached to an inserter.

FIG. 7A is another variation of a stabilization device connected to an inserter. FIGS. 7B and 7C show detail of the distal and proximal ends (respectively) of the stabilization device and inserter of FIG. 7A.

FIG. 8A is one variation of a handle that may be used with an inserter.

FIGS. 8B-8E illustrate connecting an inserter to a handle such as the handle of FIG. 8A.

FIGS. 9A-9D illustrate the operation of an inserter and handle in converting a stabilization device from a relaxed, deployed configuration (in FIGS. 9A and 9B) to a contracted, delivery configuration (in FIGS. 9C and 9D).

FIG. 10 is one variation of an inserter connected to a stabilization device within an access cannula.

FIG. 11 shows one variation of a trocar and access cannula.

FIG. 12A-12C shows one variation of a hand drill.

FIG. 13 shows one variation of a cement cannula and two cement filling devices.

FIGS. 14A-14D show different variations of an access cannula that may be used with a stabilization device and inserter, trocar, drill, and cement cannula, respectively.

FIGS. 15A-15G illustrate one method of treating a bone.

FIGS. 16A-16B illustrate one method of using bone cement with the stabilization devices described herein.

FIG. 17 is a schematic flowchart illustrating one method of treating a bone using the stabilization devices described herein.

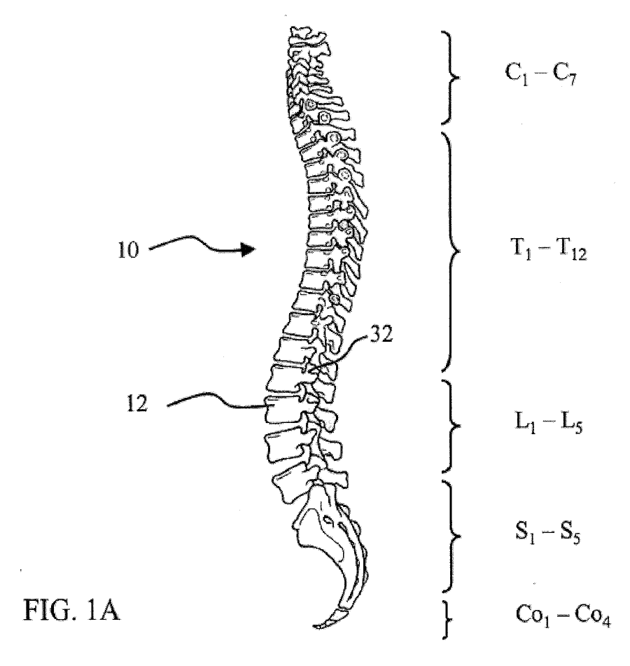

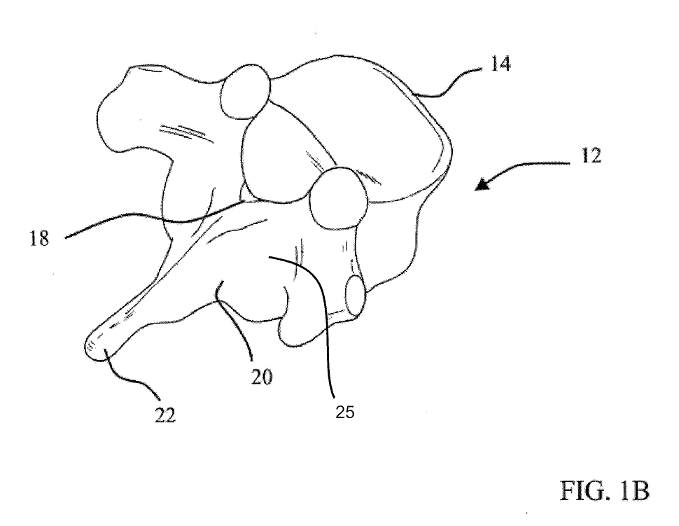

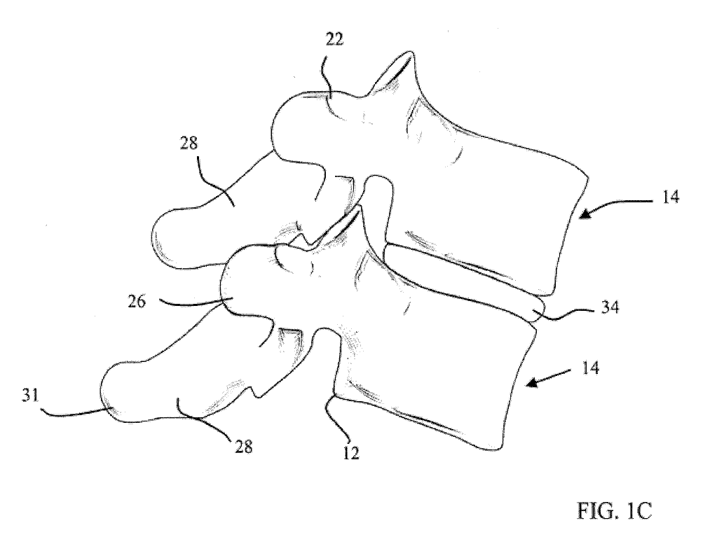

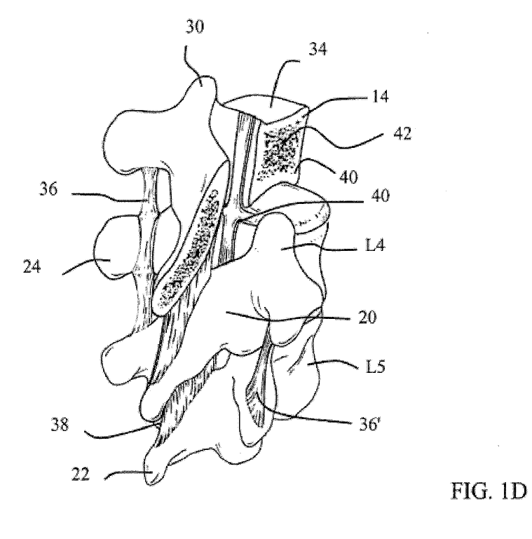

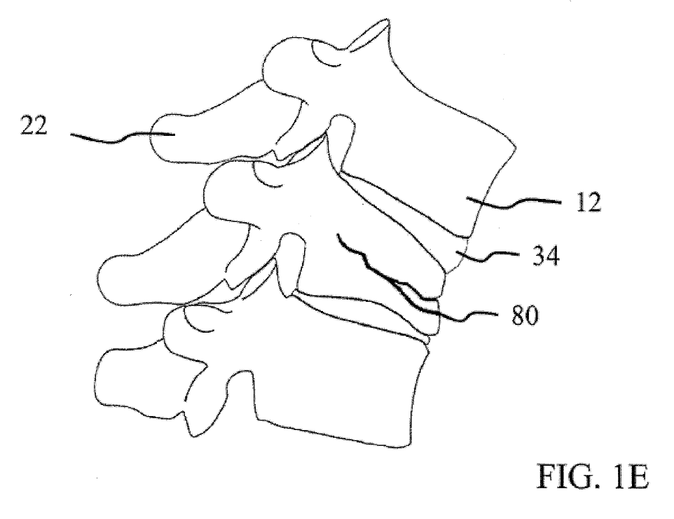

### DETAILED DESCRIPTION OF THE INVENTION

The devices, systems and methods described herein may aid in the treatment of fractures and microarchitetcture deterioration of bone tissue, particularly vertebral compression fractures (“VCFs”). The implantable stabilization devices described herein (which may be referred to as “stabilization devices” or simply “devices”) may help restore and/or augment bone. Thus, the stabilization devices described herein may be used to treat pathologies or injuries. For purposes of illustration, many of the devices, systems and methods described herein are shown with reference to the spine. However, these devices, systems and methods may be used in any appropriate body region, particularly bony regions. For example, the methods, devices and systems described herein may be used to treat hip bones.

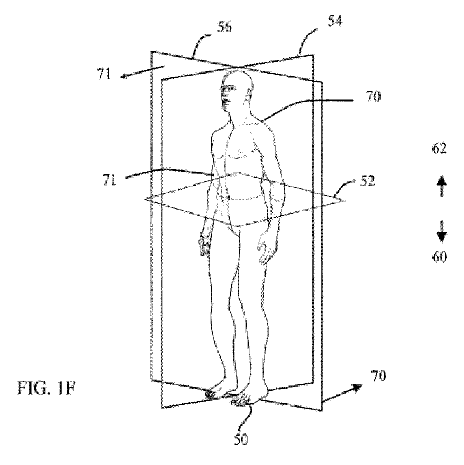

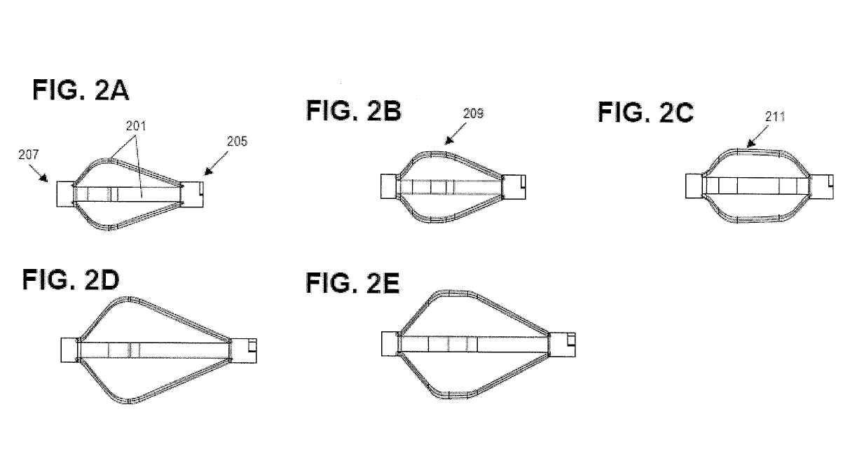

The stabilization devices described herein may be self-expanding devices that expand from a compressed profile having a relatively narrow diameter (e.g., a delivery configuration) into an expanded profile (e.g., a deployed configuration). The stabilization devices generally include a shaft region having a plurality of struts that may extend from the shaft body. The distal and proximal regions of a stabilization device may include one or more attachment regions configured to attach to an inserter for inserting (and/or removing) the stabilization device from the body. FIGS. 2A through 6 show exemplary stabilization devices.

Side profile views of five variations of stabilization devices are shown in FIGS. 2A through 2E. FIG. 2A shows a 10 mm asymmetric stabilization device in an expanded configuration. The device has four struts 201, 201′, formed by cutting four slots down the length of the shaft. In this example, the elongate expandable shaft has a hollow central lumen, and a proximal end 205 and a distal end 207. By convention, the proximal end is the end closest to the person inserting the device into a subject, and the distal end is the end furthest away from the person inserting the device.

The struts 201, 201′ of the elongate shaft is the section of the shaft that projects from the axial (center) of the shaft. Three struts are visible in each of FIGS. 2A-2E. In general, each strut has a leading exterior surface that forms a cutting surface adapted to cut through cancellous bone as the strut is expanded away from the body of the elongate shaft. This cutting surface may be shaped to help cut through the cancellous bone (e.g., it may have a tapered region, or be sharp, rounded, etc.). In some variations, the cutting surface is substantially flat.

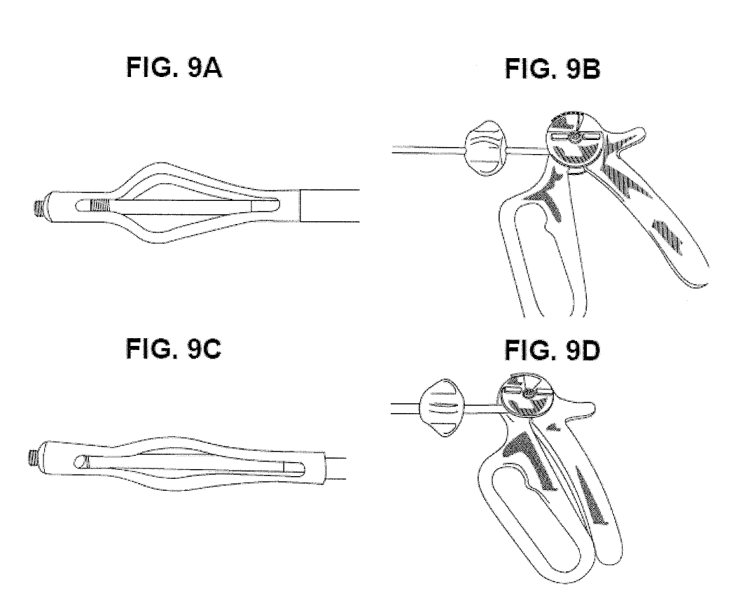

The stabilization device is typically biased so that it is relaxed in the expanded or deployed configuration, as shown in FIGS. 2A to 2E. In general, force may be applied to the stabilization device so that it assumes the narrower delivery profile, described below (and illustrated in FIG. 9C). Thus, the struts may elastically bend or flex from the extended configuration to the unextended configuration.

The struts in all of these examples are continuous curvature of bending struts. Continuous curvature of bending struts are struts that do not bend from the extended to an unextended configuration (closer to the central axis of the device shaft) at a localized point along the length of the shaft. Instead, the continuous curvature of bending struts are configured so that they translate between a delivery and a deployed configuration by bending over the length of the strut rather than by bending at a discrete portion (e.g., at a notch, hinge, channel, or the like). Bending typically occurs continuously over the length of the strut (e.g., continuously over the entire length of the strut, continuously over the majority of the length of the strut (e.g., between 100-90%, 100-80%, 100-70%, etc.), continuously over approximately half the length of the strut (e.g., between about 60-40%, approximately 50%, etc.).

The “curvature of bending” referred to by the continuous curvature of bending strut is the curvature of the change in configuration between the delivery and the deployed configuration. The actual curvature along the length of a continuous curvature of bending strut may vary (and may even have “sharp” changes in curvature). However, the change in the curvature of the strut between the delivery and the deployed configuration is continuous over a length of the strut, as described above, rather than transitioning at a hinge point. Struts that transition between delivery and deployed configurations in such a continuous manner may be stronger than hinged or notched struts, which may present a pivot point or localized region where more prone to structural failure.

Thus, the continuous curvature of bending struts do not include one or more notches or hinges along the length of the strut. Two variations of continuous curvature of bending struts are notchless struts and/or hingeless struts. In FIG. 2A, the strut 201 bends in a curve that is closer to the distal end of the device than the proximal end (making this an asymmetric device). In this example, the maximum distance between the struts along the length of device is approximately 10 mm in the relaxed (expanded) state. Thus, this may be referred to as a 10 mm asymmetric device.

FIG. 2B shows another example of a 10 mm asymmetric device in which the curve of the continuous curvature of bending strut has a more gradual bend than the devices shown in FIG. 2A. This variation may be particularly useful when the device is used to support non-cancellous bone in the deployed state. For example, the flattened curved region 209 of the continuous curvature of bending strut may provide a contact surface to support the non-cancellous bone. For example, the leading edge of the strut (the cutting edge) may expand through the cancellous bone and abut the harder cortical bone forming the exterior shell of the bony structure. FIG. 2C shows a symmetric 10 mm device in which this concept 211 is even more fully developed. FIGS. 2D and 2E are examples of 18 mm devices similar to the 10 mm devices shown in FIGS. 2A and 2B, respectively.

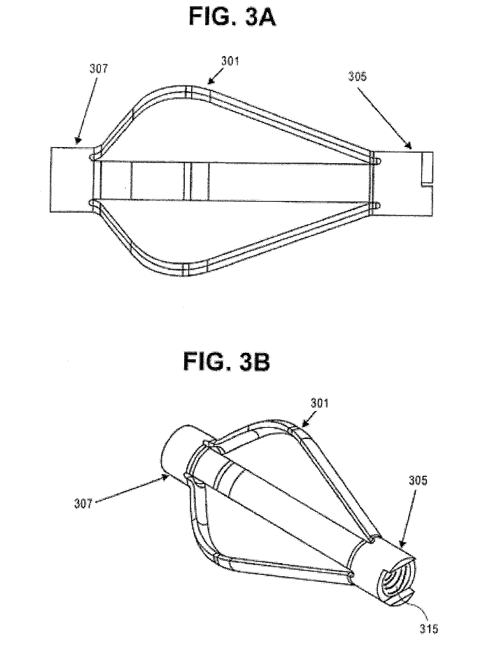

FIGS. 3A and 3B show enlarged side and side perspective views (respectively) of the 10 mm asymmetric device shown in FIG. 2A. These figures help further illustrate the continuous curve of the continuous curvature of bending strut 301. The proximal end (the end facing to the right in FIGS. 3A and 3B), shows one variation of an attachment region to which the device may be attached to one portion of an introducer. In this example, the end includes a cut-out region 305, forming a seating area into which a complementary attachment region of an inserter may mate. Although not visible in FIGS. 3A and 3B, the distal region 307 of the device may also include an attachment region. In some variations, the inner region (and/or outer region) of the proximal end 315 of the device may be threaded. Threads may also be used to engage the inserter at the proximal (and/or distal) ends of the device as part of the attachment region.

An attachment region may be configured in any appropriate way. For example, the attachment region may be a cut-out region (or notched region), including an L-shaped cut out, an S-shaped cut out, a J-shaped cut out, or the like, into which a pin, bar, or other structure on the inserter may mate. In some variations, the attachment region is a threaded region which may mate with a pin, thread, screw or the like on the inserter. In some variations, the attachment region is a hook or latch. The attachment region may be a hole or pit, with which a pin, knob, or other structure on the inserter mates. In some variations, the attachment region includes a magnetic or electromagnetic attachment (or a magnetically permeable material), which may mate with a complementary magnetic or electromagnet region on the inserter. In each of these variations the attachment region on the device mates with an attachment region on the inserter so that the device may be removably attached to the inserter.

The stabilization devices described herein generally have two or more releasable attachment regions for attaching to an inserter. For example, a stabilization device may include at least one attachment region at the proximal end of the device and another attachment region at the distal end of the device. This may allow the inserter to apply force across the device (e.g., to pull the device from the expanded deployed configuration into the narrower delivery configuration), as well as to hold the device at the distal end of the inserter. However, the stabilization devices may also have a single attachment region (e.g., at the proximal end of the device). In this variation, the more distal end of the device may include a seating region against which a portion of the inserter can press to apply force to change the configuration of the device. In some variations of the self-expanding stabilization devices, the force to alter the configuration of the device from the delivery to the deployed configuration comes from the material of the device itself (e.g., from a shape-memory material), and thus only a single attachment region (or one or more attachment region at a single end of the device) is necessary.

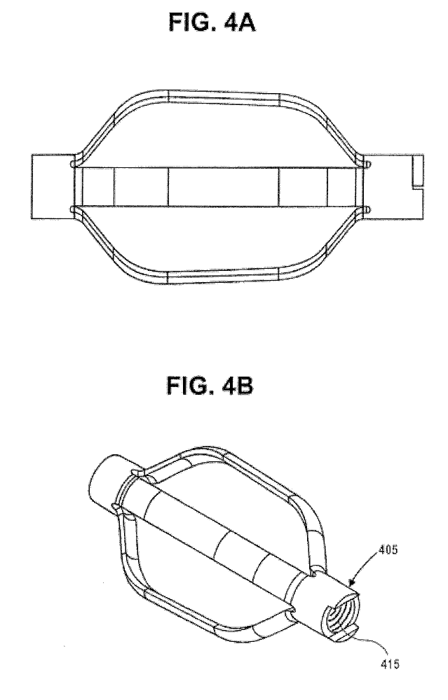

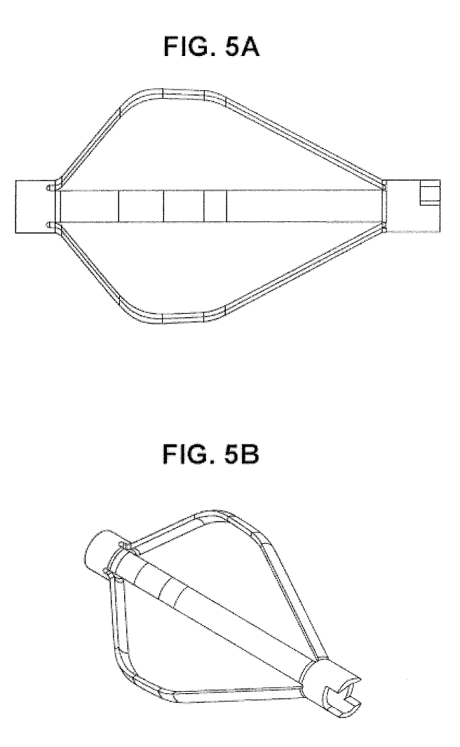

Similar to FIGS. 3A and 3B, FIGS. 4A and 4B show side and side perspective views of exemplary symmetric 10 mm devices, and FIGS. 5A and 5B show side and side perspective views of 18 mm asymmetric devices.

The continuous curvature of bending struts described herein may be any appropriate dimension (e.g., thickness, length, width), and may have a uniform cross-sectional thickness along their length, or they may have a variable cross-sectional thickness along their length. For example, the region of the strut that is furthest from the tubular body of the device when deployed (e.g., the curved region 301 in FIGS. 3A and 3B) may be wider than other regions of the strut, providing an enhanced contacting surface that abuts the non-cancellous bone after deployment.

The dimensions of the struts may also be adjusted to calibrate or enhance the strength of the device, and/or the force that the device exerts to self-expand. For example, thicker struts (e.g., thicker cross-sectional area) may exert more force when self-expanding than thinner struts. This force may also be related to the material properties of the struts.

The struts may be made of any appropriate material. In some variations, the struts and other body regions are made of substantially the same material. Different portions of the stabilization device (including the struts) may be made of different materials. In some variations, the struts may be made of different materials (e.g., they may be formed of layers, and/or of adjacent regions of different materials, have different material properties). The struts may be formed of a biocompatible material or materials. It may be beneficial to form struts of a material having a sufficient spring constant so that the device may be elastically deformed from the deployed configuration into the delivery configuration, allowing the device to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol), tantalum, tantalum alloys, aluminum, etc. Any appropriate shape memory material, including shape memory alloys such as Nitinol may also be used.

Other regions of the stabilization device may be made of the same material(s) as the struts, or they may be made of a different material. Any appropriate material (preferably a biocompatible material) may be used (including any of those materials previously mentioned), such as metals, plastics, ceramics, or combinations thereof. In variations where the devices have bearing surfaces (i.e. surfaces that contact another surface), the surfaces may be reinforced. For example, the surfaces may include a biocompatible metal. Ceramics may include pyrolytic carbon, and other suitable biocompatible materials known in the art. Portions of the device can also be formed from suitable polymers include polyesters, aromatic esters such as polyalkylene terephthalates, polyamides, polyalkenes, poly(vinyl) fluoride, PTFE, polyarylethyl ketone, and other materials. Various alternative embodiments of the devices and/or components could comprise a flexible polymer section (such as a biocompatible polymer) that is rigidly or semi rigidly fixed.

The devices (including the struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the stabilization devices may elute a bioactive substance to promote or inhibit bone growth, vascularization, etc. In one variation, the device includes an elutible reservoir of bone morphogenic protein (BMP).

As previously mentioned, the stabilization devices may be formed about a central elongate hollow body. In some variations, the struts are formed by cutting a plurality of slits long the length (distal to proximal) of the elongate body. This construction may provide one method of fabricating these devices, however the stabilization devices are not limited to this construction. If formed in this fashion, the slits may be cut (e.g., by drilling, laser cutting, etc.) and the struts formed by setting the device into the deployed shape so that this configuration is the default, or relaxed, configuration in the body. For example, the struts may be formed by plastically deforming the material of the struts into the deployed configuration. In general, any of the stabilization devices may be thermally treated (e.g., annealed) so that they retain this deployed configuration when relaxed. Thermal treatment may be particularly helpful when forming a strut from a shape memory material such as Nitinol into the deployed configuration.

### Inserter

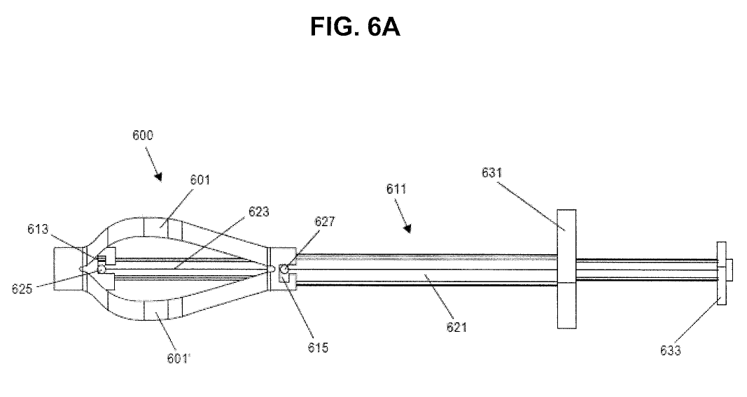

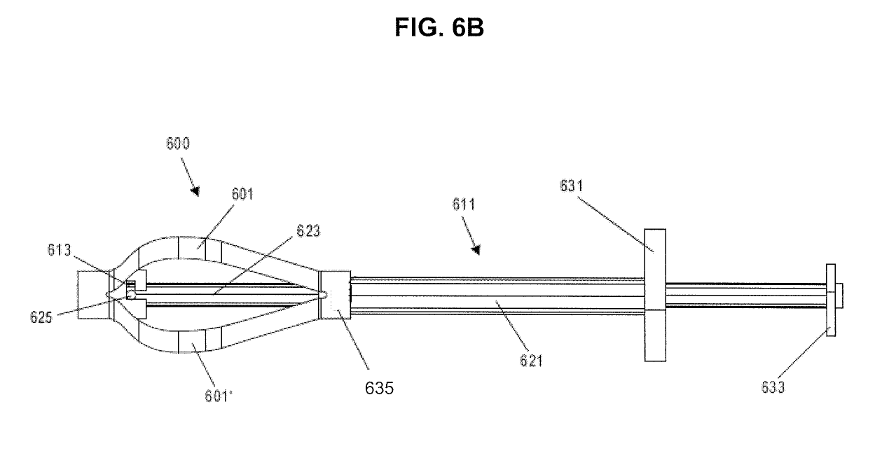

FIG. 6A shows a stabilization device 600 having a plurality of continuous curvature of bending struts 601, 601′ removably attached to an inserter 611. In this example, an attachment region 615 at the proximal portion of the stabilization device is configured as an L-shaped notch, as is the attachment region 613 at the distal portion of the device.

In general, an inserter includes an elongate body having a distal end to which the stabilization device may be attached and a proximal end which may include a handle or other manipulator that coordinates converting an attached stabilization device from a delivery and a deployed configuration, and also allows a user to selectively release the stabilization device from the distal end of the inserter.

The inserter 611 shown in FIG. 6A includes a first elongate member 621 that coaxially surrounds a second elongate member 623. In this variation, each elongate member 621, 623 includes a stabilization device attachment region at its distal end, to which the stabilization device is attached, as shown. In this example, the stabilization device attachment region includes a pin that mates with the L-shaped slots forming the releasable attachment regions on the stabilization device. In FIG. 6A the L-shaped releasable attachments on the stabilization device are oriented in opposite directions (e.g., the foot of each “L” points in opposite directions). Thus, the releasable attachment devices may be locked in position regardless of torque applied to the inserter, preventing the stabilization device from being accidentally disengaged.

The inserter shown in FIG. 6A also includes two grips 631, 633 at the proximal ends of each elongate member 621, 623. These grips can be used to move the elongate members (the first 621 or second 623 elongate member) relative to each other. The first and second elongate members of the inserter may be moved axially (e.g., may be slid along the long axis of the inserter) relative to each other, and/or they may be moved in rotation relative to each other (around the common longitudinal axis). Thus, when a stabilization device is attached to the distal end of the inserter, moving the first elongate member 621 axially with respect to the second elongate member 623 will cause the stabilization device to move between the deployed configuration (in which the struts are expanded) and the delivery configuration (in which the struts are relatively unexpanded). Furthermore, rotation of the first elongate member of the inserter relative to the second elongate member may also be used to disengage one or more releasable attachment regions of the stabilization device 613, 615 from the complementary attachment regions of the inserter 625, 627. Although he stabilization devices described herein are typically self-expanding stabilization devices, the inserter may be used with stabilization devices that do not self-expand. Even in self-expanding devices, the inserter may be used to apply additional force to convert the stabilization device between the delivery and the deployed configuration. For example, when allowed to expand in a cancellous bone, the force applied by the struts when self-expanding may not be sufficient to completely cut through the cancellous bone and/or distract the cortical bone as desired. In some variations, the inserter may also permit the application of force to the stabilization device to expand the struts even beyond the deployed configuration.

An inserter may also limit or guide the movement of the first and second elongate members, so as to further control the configuration and activation of the stabilization device. For example, the inserter may include a guide for limiting the motion of the first and second elongate members. A guide may be a track in either (or both) elongate member in which a region of the other elongate member may move. The inserter may also include one or more stops for limiting the motion of the first and second elongate members.

As mentioned above, the attachment regions on the inserter mate with the stabilization device attachments. Thus, the attachment regions of the inserter may be complementary attachments that are configured to mate with the stabilization device attachments. For example, a complimentary attachment on an inserter may be a pin, knob, or protrusion that mates with a slot, hole, indentation, or the like on the stabilization device. The complementary attachment (the attachment region) of the inserter may be retractable. For example, the inserter may include a button, slider, etc. to retract the complementary attachment so that it disconnects from the stabilization device attachment. A single control may be used to engage/disengage all of the complementary attachments on an inserter, or they may be controlled individually or in groups.

FIG. 6B is another variation of a stabilization device 600 releasably connected to an inserter 61 1, in which the attachment region 635 between the stabilization device and the inserter is configured as a screw or other engagement region, rather than the notch 615 shown in FIG. 6A.

In some variation the inserter includes a lock or locks that hold the stabilization device in a desired configuration. For example, the inserter may be locked so that the stabilization device is held in the delivery configuration (e.g., by applying force between the distal and proximal ends of the stabilization device). In an inserter such as the one shown in FIG. 6A, for example, a lock may secure the first elongate member to the second elongate member so that they may not move axially relative to each other.

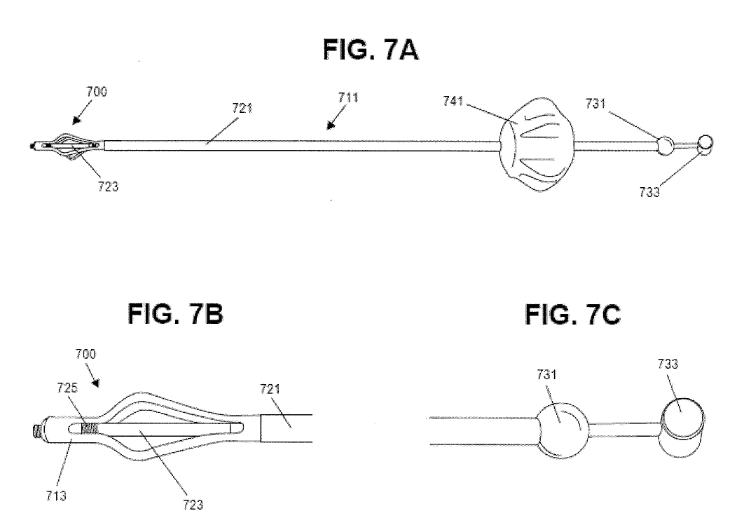

FIG. 7A is another example of an inserter 711 and an attached stabilization device 700. Similar to FIG. 6A, the stabilization device includes a first elongate member 721 attached to the proximal end of the stabilization device, and a second elongate member 723 attached to the distal end of the stabilization device. The first 721 and the second 723 elongate members are also configured coaxially (as a rod and shaft) that may be moved axially and rotationally independently of each other. The stabilization device 700 includes a plurality of continuous curvature of bending struts, shown in detail in FIG. 7B. The stabilization device 700 is shown in the deployed configuration. The distal end of the stabilization device includes a releasable attachment 713 that is configured as a threaded region which mates with a threaded complementary attachment 725 at the distal end of the structure.

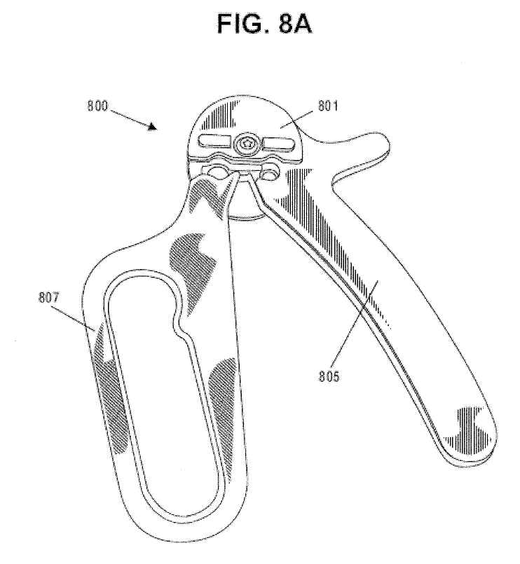

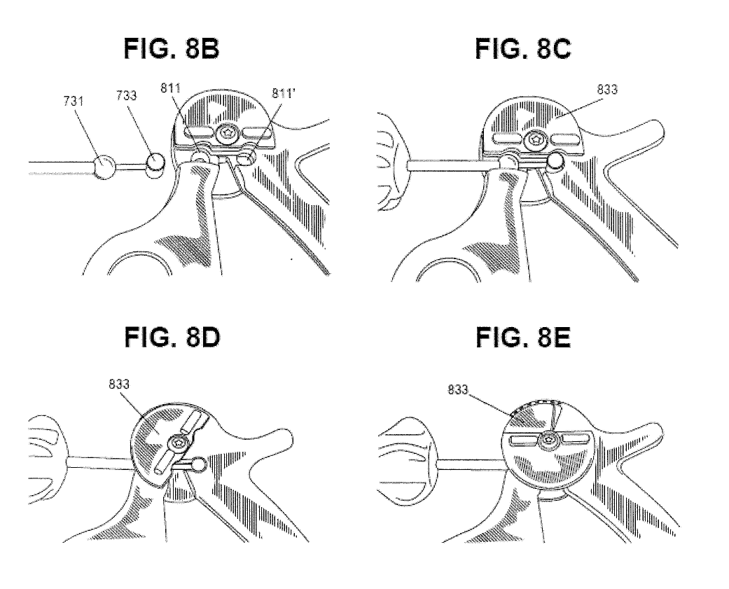

The proximal ends of the coaxial first and second elongated members 721, 723 also include grips 731, 733. These grips are shown in greater detail in FIG. 7C. As with the grips described in FIG. 6A, these grips may be grasped directly by a person (e.g., a physician, technician, etc.) using the device, or they may be connected to a handle. Thus, in some variations one or both grips are ‘keyed’ to fit into a handle, so that they can be manipulated by the handle. An example of this is shown in FIG. 8A-8E, and described below. The inserter of FIG. 7A also includes a knob 741 attached to the first elongated member 721 distal to the proximal end of the elongated member. This knob may also be used to move the first (or outer) elongate member of the inserter (e.g., to rotate it), or to otherwise hold it in a desired position. The knob may be shaped and/or sized so that it may be comfortably handheld.

Any of the inserters described herein may include, or may be used with, a handle. A handle may allow a user to control and manipulate an inserter. For example, a handle may conform to a subject's hand, and may include other controls, such as triggers or the like. Thus, a handle may be used to control the relative motion of the first and second elongate members of the inserter, or to release the connection between the stabilization device and the inserter, or any of the other features of the inserter described herein.

An inserter may be packaged or otherwise provided with a stabilization device attached. Thus, the inserter and stabilization device may be packaged sterile, or may be sterilizable. In some variations, a reusable handle is provided that may be used with a pre-packaged inserter stabilization device assembly. In some variations the handle is single-use or disposable. The handle may be made of any appropriate material. For example, the handle may be made of a polymer such as polycarbonate.

FIG. 8A illustrates one variation of a handle 800 that may be used with an inserter, such as the inserter shown in FIGS. 7A-7C. The handle 800 includes a hinged joint 803, and the palm contacting 805 region and finger contacting 807 region of the handle 800 may be moved relative to each other by rotating about this hinged joint 803. This variation of a handle also includes a thumb rest 809, which may also provide additional control when manipulating an inserter with the handle. The thumb rest may also include a button, trigger, or the like.

FIGS. 8B-8E illustrate the connection of an inserter such as the inserter described above in FIGS. 7A-C into a handle 800. In FIG. 8B the proximal end of the inserter is aligned with openings 811, 811′ in the handle. These openings are configures so that the grips 731, 733 at the distal ends of the first and second elongate members of the inserter can fit into them. In this example, the grip 733 is shaped so that it can be held in the opening 811′ of the handle in an oriented fashion, preventing undesirable rotation. Thus, in FIG. 8C the proximal end of the inserter (the grips 731 and 732) are placed in the openings 811, 811′. The inserter may then be secured to the handle by rotating cover 833, as shown in FIGS. 8D and 8E.

By securing the proximal end of the inserter in the handle, the handle can then be used to controllably actuate the inserter, as illustrated in FIGS. 9A-9D. In this example the stabilization device is in the deployed configuration (shown in FIG. 9A) when the handle is “open” (shown in FIG. 9B). By squeezing the handle (rotating the finger grip region towards the palm region, as shown in FIG. 9D) the inserter applies force between the proximal and distal regions of the stabilization device, placing it in a delivery configuration, as shown in FIG. 9C.

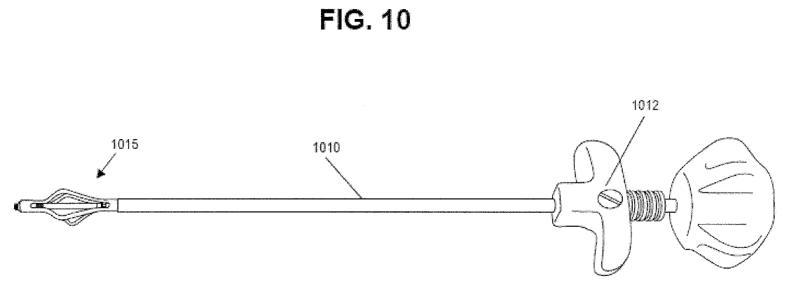

As mentioned above, in the delivery configuration the struts of the stabilization device are typically closer to the long axis of the body of the stabilization device. Thus, the device may be inserted into the body for delivery into a bone region. This may be accomplished with the help of an access cannula (which may also be referred to as an introducer). As shown in FIG. 10, the inserter 1015 is typically longer than the access cannula 1010, allowing the stabilization device to project from the distal end of the access cannula for deployment. The access cannula may also include a handle 1012.

Any of the devices (stabilization devices) and inserters (including handles) may be included as part of a system or kit for correcting a bone defect or injury. FIGS. 10 through 14D illustrate different examples of tools (or variations of tools) that may be used as part of a system for repair bone. Any of these tools (or additional tools) may also be used to perform the methods of repairing bone (particularly spinal bone) described herein. For example, FIG. 11 shows a trocar 1105 having a handle 1107 and a cutting/obdurating tip 1109. This trocar 1105 may also be used with an access cannula 1111. Another example of an access cannula 1111 (or introducer) is shown adjacent to the trocar 1106 in FIG. 11. This exemplary access cannula has an inner diameter of approximately 4.2 mm, so that the trocar 1105 will fit snugly within it, and a stabilization device in a delivery configuration will also fit therein. Any appropriate length cannula and trocar may be used, so long as it is correctly scaled for use with the introducer and stabilization device. For example, the access cannula may be approximately 15.5 cm long. The trocar an introducer may be used to cut through tissue until reaching bone, so that the introducer can be positioned appropriately.

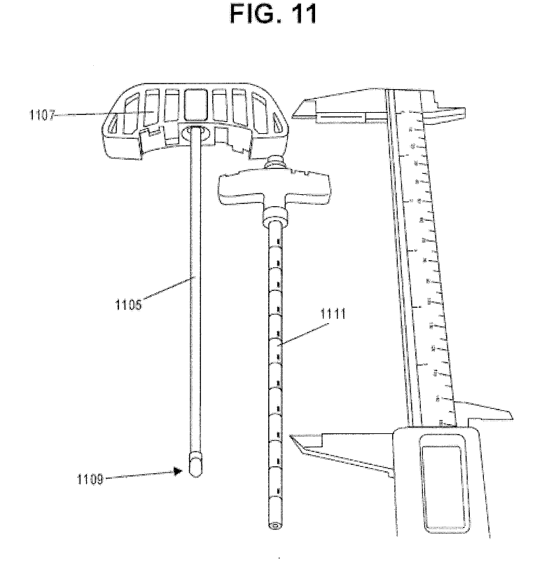

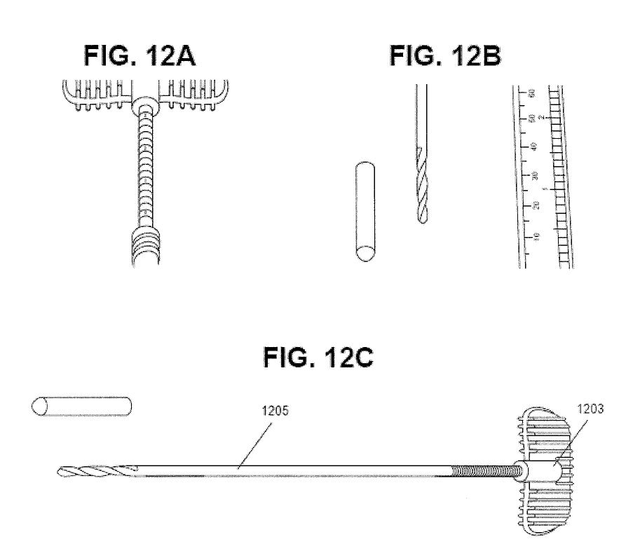

A bone drill, such as the hand drill shown in FIGS. 12A-12C, may then be used to access the cancellous bone. The twist drill 1201 shown in FIG. 12A-12C has a handle 1203 at the proximal end and a drill tip 1205 at the distal end. This twist drill may be used with the same access cannula previously described (e.g., in this example the twist drill has an outer diameter of 4.1 mm and a length of 19.5 cm). The distal (drill) end of the twist drill may extend from the cannula, and be used to drill into the bone. The proximal end of the twist drill shown in FIGS. 12A-12C is calibrated (or graduated) to help determine the distance drilled.

Any of the devices shown and described herein may also be used with a bone cement. For example, a bone cement may be applied after inserting the stabilization device into the bone, positioning and expanding the device (or allowing it to expand and distract the bone) and removing the inserter, leaving the device within the bone. Bone cement may be used to provide long-term support for the repaired bone region.

Any appropriate bone cement or filler may be used, including PMMA, bone filler or allograft material. Suitable bone filler material include bone material derived from demineralized allogenic or xenogenic bone, and can contain additional substances, including active substance such as bone morphogenic protein (which induce bone regeneration at a defect site). Thus materials suitable for use as synthetic, non-biologic or biologic material may be used in conjunction with the devices described herein, and may be part of a system includes these devices. For example, polymers, cement (including cements which comprise in their main phase of microcrystalline magnesium ammonium phosphate, biologically degradable cement, calcium phosphate cements, and any material that is suitable for application in tooth cements) may be used as bone replacement, as bone filler, as bone cement or as bone adhesive with these devices or systems. Also included are calcium phosphate cements based on hydroxylapatite (HA) and calcium phosphate cements based on deficient calcium hydroxylapatites (CDHA, calcium deficient hydroxylapatites). See, e.g., U.S. Pat. No. 5,405,390 to O'Leary et al.; U.S. Pat. No. 5,314,476 to Prewett et al.; U.S. Pat. No. 5,284,655 to Bogdansky et al.; U.S. Pat. No. 5,510,396 to Prewett et al.; U.S. Pat. No. 4,394,370 to Jeffries; and U.S. Pat. No. 4,472,840 to Jeffries, which describe compositions containing demineralized bone powder. See also U.S. Pat. No. 6,340,477 to Anderson which describes a bone matrix composition. Each of these references is herein incorporated in their entirely.

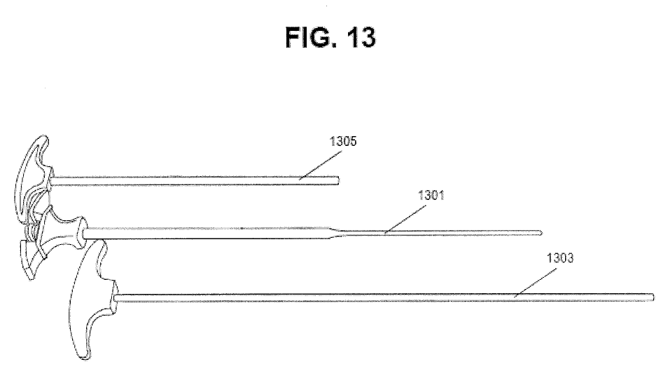

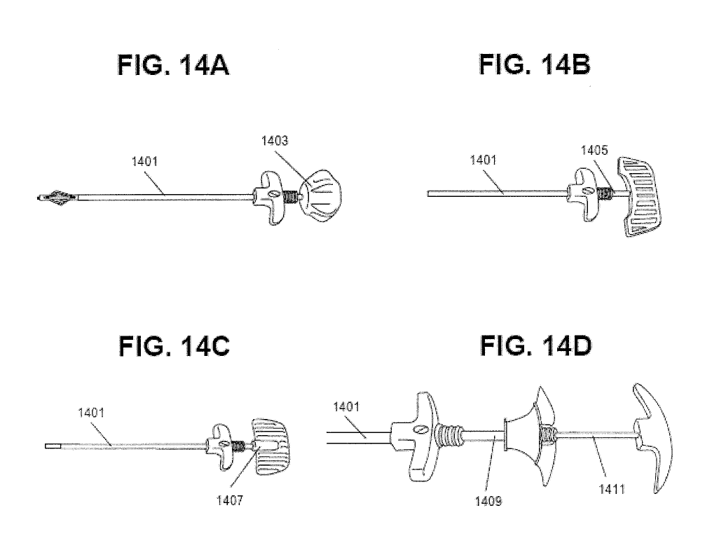

FIG. 13 shows a tapered cement cannula 1301 that may be used to deliver bone cement to the insertion site of the device, and also shows two cement obturators 1303, 1305 for delivering the cement (piston-like). The cannula delivering cement is also designed to be used through the access cannula, as are all of the components described above, including the stabilization device and inserter, trocar, and drill. This is summarized in FIGS. 14A-14D. FIG. 14A illustrates an access cannula 4101 with a stabilization device 1403 and inserter inserted through the access cannula, as shown in FIG. 10. FIG. 14B shows a trocar 1405 within the access cannula 1401. FIG. 14C shows a hand drill 1407 within the same access cannula 1401, and FIG. 14D shows a cement cannula 1409 and a cement obturator 1411 within the same access cannula 1401. These devices may be used to repair a bone.

### Exemplary Method of Repairing a Bone

As mentioned above, any of the devices described herein may be used to repair a bone. A method of treating a bone using the devices describe herein typically involves delivering a stabilization device (e.g., a self-expanding stabilization device as described herein) within a cancellous bone region, and allowing the device to expand within the cancellous bone region so that a cutting surface of the device cuts through the cancellous bone.

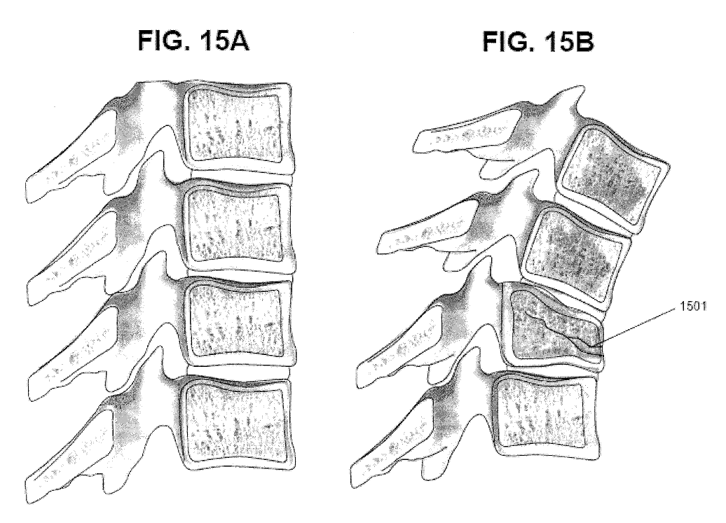

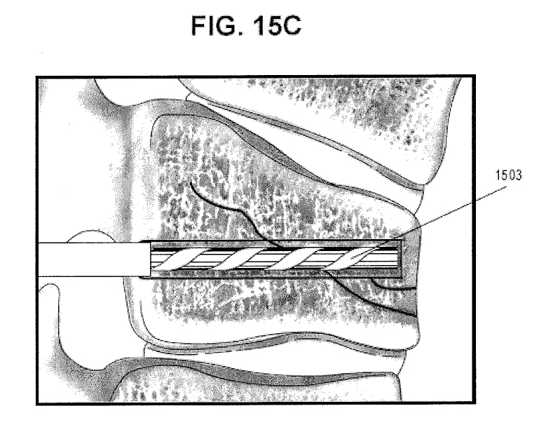

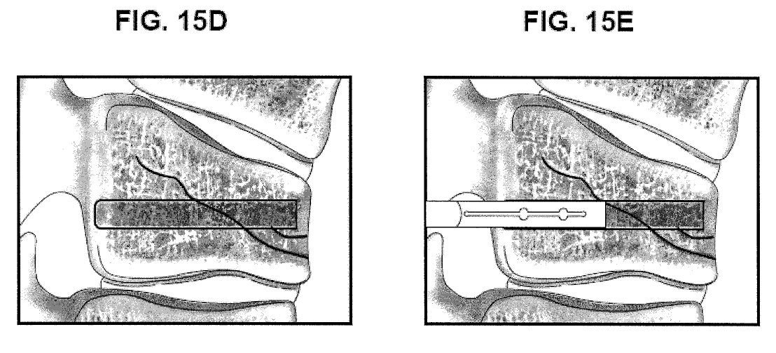

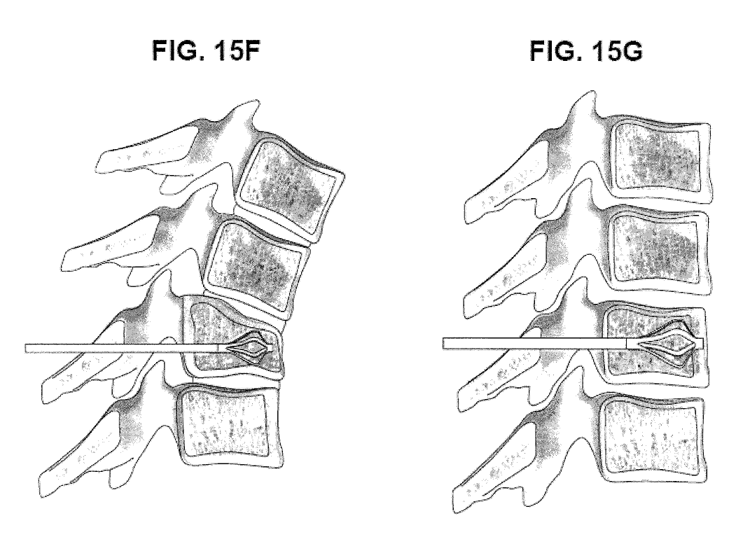

For example, the stabilization devices described herein may be used to repair a compression fracture in spinal bone. This is illustrated schematically in FIGS. 15A-15G. FIG. 15A shows a normal thoracic region of the spine in cross-section along the sagital plane. The spinal vertebra are aligned, distributing pressure across each vertebra. FIG. 15B shows a similar cross-section through the spine in which there is a compression fracture in the 11^{th} thoracic vertebra 1501. The 11^{th} vertebra is compressed in the fractured region. It would be beneficial to restore the fractured vertebra to its uninjured position, by expanding (also referred to as distracting) the vertebra so that the shape of the cortical bone is restored. This may be achieved by inserting and expanding one of the stabilization devices described herein. In order to insert the stabilization device, the damaged region of bone must be accessed.

As mentioned above, an introducer (or access cannula) and a trocar, such as those shown in FIG. 11 may be used to insert the access cannula adjacent to the damaged bone region. Any of the steps described herein may be aided by the use of an appropriate visualization technique. For example, a fluoroscope may be used to help visualize the damaged bone region, and to track the p of inserting the access cannula, trocar, and other tools. Once the access cannula is near the damaged bone region, a bone drill may be used to drill into the bone, as shown in FIG. 15C.

In FIG. 15C the drill 1503 enters the bone from the access cannula. The drill enters the cancellous bony region within the vertebra. After drilling into the vertebra to provide access, the drill is removed from the bone and the access cannula is used to provide access to the damaged vertebra, as shown, by leaving the access cannula in place, providing a space into which the stabilization device may be inserted in the bone, as shown in FIG. 15D. In FIG. 15E a stabilization device, attached to an inserter and held in the delivery configuration, is inserted into the damaged vertebra.

Once in position within the vertebra, the stabilization device is allowed to expand (by self-expansion) within the cancellous bone of the vertebra, as shown in FIG. 15F. In some variations, the device may fully expand, cutting through the cancellous bone and pushing against the cortical bone with a sufficient restoring force to correct the compression, as shown in FIG. 15G. However, in some variations, the force generated by the device during self-expansion is not sufficient to distract the bone, and the inserter handle may be used (e.g., by applying force to the handle, or by directly applying force to the proximal end of the inserter) to expand the stabilization device until the cortical bone is sufficiently distracted.

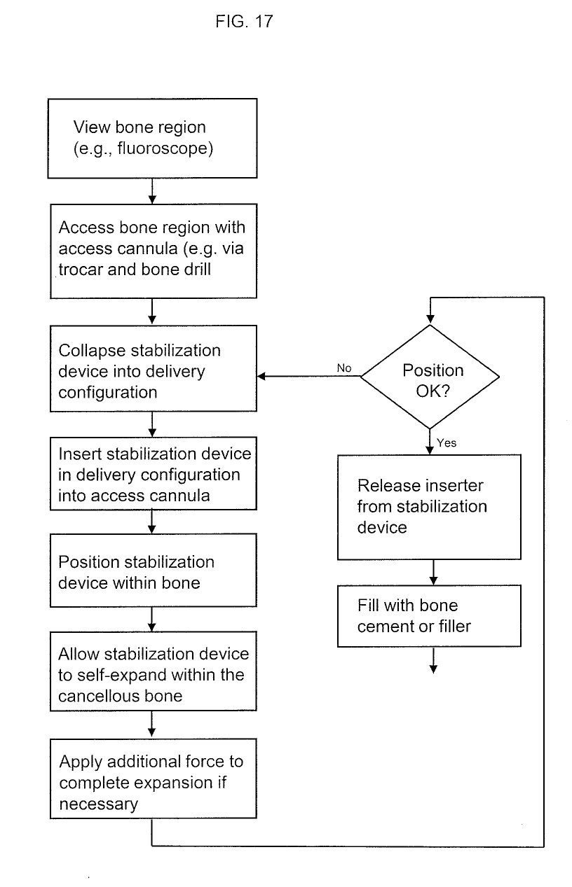

Once the stabilization device has been positioned and is expanded, it may be released from the inserter. In some variations, it may be desirable to move or redeploy the stabilization device, or to replace it with a larger or smaller device. If the device has been separated from the inserter (e.g., by detaching the removable attachments on the stabilization device from the cooperating attachments on the inserter), then it may be reattached to the inserter. Thus, the distal end of the inserter can be coupled to the stabilization device after implantation. The inserter can then be used to collapse the stabilization device back down to the delivery configuration (e.g., by compressing the handle in the variation shown in FIGS. 9A-9D), and the device can be withdrawn or re-positioned. FIG. 17 shows a flowchart summarizing a method for repairing a bone, as described herein.

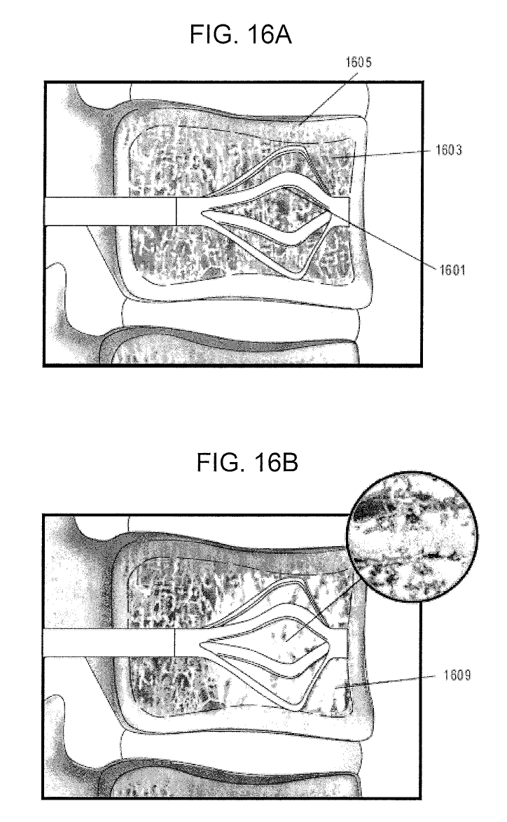

As mentioned above, a cement or additional supporting material may also be used to help secure the stabilization device in position and repair the bone. For example, bone cement may be used to cement a stabilization device in position. FIGS. 16A-16C illustrate one variation of this. In FIG. 16A the stabilization device 1601 has been expanded within the cancellous bone 1603 and is abutting the cortical bone 1605. Although in some variations the addition of the stabilization device may be sufficient to repair the bone, it may also be desirable to add a cement, or filler to help secure the repair. This may also help secure the device in position, and may help close the surgical site.

For example, in FIG. 16B a fluent bone cement 1609 has been added to the cancellous bone region around implant. This cement will flow through the channels of trebeculated (cancellous) bone, and secure the implant in position. This is shown in greater detail in the enlarged region shown in FIG. 16C. This bone cement or filler can be applied using the delivery cannula (e.g., through a cement cannula, as described above), and allowed to set.

While preferred embodiments of the present invention have been shown and described herein, such embodiments are provided by way of example only. Numerous variations, changes, and substitutions are possible without departing from the invention. Thus, alternatives to the embodiments of the invention described herein may be employed in practicing the invention. The exemplary claims that follow help further define the scope of the systems, devices and methods (and equivalents thereof).

What may be claimed is:

1. A stabilization device configured to self-expand from a compressed delivery configuration to an expanded deployed configuration, the stabilization device comprising:
an elongate shaft having two or more continuous curvature of bending struts, wherein the struts extend from the shaft more in the deployed configuration than in the delivery configuration;
a proximal region having a first releasable attachment configured to attach to an inserter; and
a distal region having a second releasable attachment configured to attach to the inserter.

2. The device of claim 1, further comprising two or more slits in the elongate shaft.

3. The device of claim 2, wherein the struts are formed by two or more slits.

4. The device of claim 1, wherein the struts are formed of a shape memory alloy.

5. The device of claim 3, wherein the shape memory alloy is Nitinol.

6. The device of claim 1, wherein the first releasable attachment comprises an L-shaped notch.

7. The device of claim 1, wherein the first releasable attachment comprises a threaded region.

8. The device of claim 1, wherein the second releasable attachment comprises an L-shaped notch.

9. The device of claim 1, wherein the maximum distance between the struts at a point along the length of the shaft in the expanded deployed configuration is between about 0.5 and about 30 mm.

10. The device of claim 1, wherein the maximum distance between the struts at a point along the length of the shaft in the expanded deployed configuration is between about 8 and about 20 mm.

11. The device of claim 1, wherein the maximum distance between the struts at a point along the length of the shaft in the expanded deployed configuration is about 10 mm.

12. The device of claim 1, wherein the maximum distance between the struts at a point along the length of the shaft in the expanded deployed configuration is about 18 mm.

13. A self-expanding stabilization device for stabilizing a body cavity, the device comprising:
an elongate shaft having a plurality of continuous curvature of bending struts extendable therefrom, the shaft adapted to be positioned within cancellous bone and having an expanded deployed profile and a collapsed delivery profile;
a proximal region configured to releasably connect to a first portion of an inserter;
a distal region configured to releasably connected to a second portion of an inserter;
wherein the shaft is adapted to cut through cancellous bone during expansion from the collapsed delivery profile to the expanded deployed profile; and
further wherein the shaft is adapted to abut a surface of cortical bone adjacent the cancellous bone without passing there through.

14. An inserter for inserting a stabilization device, the inserter comprising:
a first elongate member having a first stabilization device attachment region that is adapted to releasably attach to the proximal region of the stabilization device; and
a second elongate member having a second stabilization device attachment region that is adapted to releasably attach to the distal region of the stabilization device;
wherein the second elongate member is axially movable relative to the first elongate member.

15. The inserter of claim 14, wherein the first elongate member and the second elongate member are configured so that they may be independently rotated axially with respect to each other.

16. The inserter of claim 14, wherein the first stabilization device attachment region comprises a pin configured to mate with a channel in the proximal region of the stabilization device.

17. The inserter of claim 14, wherein the second stabilization device attachment region comprises a pin configured to mate with a channel in the distal region of the stabilization device.

18. The inserter of claim 14, further comprising a handle.

19. The inserter of claim 14, further comprising a knob on the first elongate member.

20. The inserter of claim 19, wherein the handle configured so that the inserter mates with the handle in a modular fashion.

21. The inserter of claim 14, wherein the second elongate member is coaxial to the first elongate member.

22. An inserter for inserting a stabilization device, the inserter comprising:
a first elongate member having a stabilization device attachment region at the distal end, wherein the stabilization device attachment region is adapted to releasably attach to the proximal region of the stabilization device;
a second elongate member having a stabilization device attachment region at its distal end that is adapted to releasably attach to the distal region of the stabilization device,
wherein the second elongate member is axially movable relative to the first elongate member; and the first elongate member and the second elongate member are independently axially rotatable with respect to each other;
a first handle attachment region at the proximal end of the first elongate member; and
a second handle attachment region at the proximal end of the second elongate member.

23. A system for stabilizing a vertebral body, the system comprising:
a stabilization device having an elongate shaft and a plurality of struts extending therefrom, the stabilization device configured to expand from a compressed delivery configuration to an expanded deployed configuration; and
an inserter having a first stabilization device attachment region, adapted to releasably secure to the proximal region of the stabilization device, and a second stabilization device attachment region adapted to secure to the distal region of the stabilization device.

24. The system of claim 23, further comprising an introducer.

25. The system of claim 23, further comprising a handle.

26. The system of claim 23, wherein the introducer further comprises a handle.

27. The system of claim 23, further comprising a trocar.

28. The system of claim 23, further comprising a twist drill.

29. The system of claim 23, further comprising bone cement.

30. The system of claim 23, further comprising a cement cannula.

31. A system for stabilizing a vertebral body, the system comprising any of the stabilization devices of claims 1-13 and any of the inserters of claims 14-22.

32. A method of treating a bone comprising:
delivering a self-expanding device within a cancellous bone; wherein the device has an elongate shaft and a plurality of continuous curvature of bending struts extending therefrom; and
allowing the device to expand within the cancellous bone so that a cutting surface of the device cuts through the cancellous bone.

33. The method of claim 32, further comprising visualizing the device within the bone.

34. The method of claim 32, further comprising drilling a hole into the cancellous bone through which the self-expanding device may be inserted.

35. The method of claim 32, further comprising applying force to further expand the device within the cancellous bone.

36. The method of claim 32, further comprising applying bone cement within the cancellous bone.
